(19) United States
(12) Patent Application Publication
(10) Pub. No.: US 2003/0212077 A1
(43) Pub. Date: Nov. 13, 2003

(54) Methods and compositions for treating tinea versicolor using combinations of retinoids and certain antifungals

(76) Inventor: Jay E. Birnbaum, Montville, NJ (US)

Correspondence Address: Bradley N. Ruben, PC, Suite 5A, 463 First Street, Hoboken, NJ, 07030, US

(21) Appl. No.: 10/434,049

(22) Filed: May 8, 2003

Related U.S. Application Data
(60) Provisional application No. 60/378,547, filed on May 8, 2002.

(51) Int. Cl. A61K 31/496; A61K 31/4196; A61K 31/4178; A61K 31/07; A61K 31/203

(52) U.S. Cl. 514/254.07; 514/383; 514/397; 514/559; 514/725; 514/549

(57) ABSTRACT

Tinea versicolor is a fungal infection of the skin and often results in a discoloration or dyspigmentation of the infected skin. The present invention provides as a combination therapy an antifungal for treating the fungal infection and a retinoid for correcting the skin pigmentation. Preferred antifungal agents are those which are also CYP-26 inhibitors, which delay the natural enzymatic breakdown of retinoids in the skin. Alternatively, a non-CYP-26 inhibiting antifungal can be used in combination with a retinoid, with a CYP-26 inhibitor (antifungal or not) optionally added.

### BACKGROUND OF THE INVENTION

1. The Field of the Invention

This invention relates to methods of treating tinea versicolor with combinations of retinoids and antifungals that are also, or in combination with, CYP-26 inhibitors.

2. The State of the Art

Among the common fungal infections of the skin are tinea pedis or “athlete's foot,” tinea cruris or “jock itch,” and tinea versicolor or pityriasis versicolor or “sun fungus.” The latter is caused by the fungus Malassezia furfur, first termed Pityrosporum ovale or P. orbiculare, which are now considered synonyms. Presentation of tinea versicolor is typically of hyper- or hypopigmented blotches on the skin, and the condition typically flares during warm and humid weather. Variously, hyperpigmented presentations are often diagnosed as tinea whereas hypopigmented conditions are defined as pityriasis, although it is the same infestation. Karaoui R, et al., “Tinea versicolor: ultrastructural studies on hypopigmented and hyperpigmented skin,” Dermatologica 1981;162(2):69-85.

Treatment for tinea versicolor is typically in the form of a topically-applied cream or soap, such as selenium sulfide (e.g., Selsun Blue brand), an imidazole antifungal (e.g., 2% ketoconazole, 2% miconazole, or 1% clotrimazole), tolnaftate, or sulfur-salicylic acid soaps. The imidazole antifungals are generally also known to be CYP-26 inhibitors; CYP-26 is a family of enzymes (cytochrome P450 RAI) which break down retinoids in the skin.

### SUMMARY AND OBJECTS OF THE INVENTION

In light of the foregoing, it would be beneficial to treat tinea versicolor by attacking both the infectious agent as well as correcting the hyper- and/or hypopigmentation present. Such treatment would provide an additional benefit if the same composition could be used for both hyper- and hypopigmented skin.

As such, a principle embodiment this invention provides a combination of a retinoid and an imidazole antifungal for treating tinea versicolor, wherein the imidazole is present in an amount effective to treat the underlying mycotic infestation and the retinoid is present in an amount effective to correct or normalize the pigmentation of the skin.

### DETAILED DESCRIPTION OF SPECIFIC EMBODIMENTS

It has been shown, in U.S. Pat. No. 5,750,570 and U.S. Pat. No. 6,017,960, the disclosures of which are incorporated herein by reference, that retinoids are useful in treating post-inflammatory hyperpigmentation in the dark-skinned people in which it occurs. Such treatment is effective without the use of bleaching agents or 4-hydroxyanisole. A CYP-26 inhibitor can be used with the retinoid to delay its normal degradation by the skin and thus to maintain its presence in the skin longer.

It also has been shown that retinoic acid can be used to “tan” light-skinned people, the retinoid presumably stimulating tyrosinase activity, as shown in U.S. Pat. No. 5,266,307, the disclosure of which is incorporated herein by reference.

Combining these concepts provides the basis for the present invention, the combination of a CYP-26 inhibiting imidazole antifungal and a retinoid together for topical administration. The imidazole antifungal is used to control the invasive fungus and to decrease the breakdown of retinoids in the skin. The retinoid acts to pigment or to depigment the skin (that is, to normalize the skin pigmentation), and to promote desquamation of skin cells, helping to eliminate the fungus and to normalize the appearance of the skin.

Retinoids that are likely to be useful for treating tinea (especially tinea versicolor) include natural and synthetic analogs of vitamin A (retinol), vitamin A aldehyde (retinal), vitamin A acid (retinoic acid (RA), including all-trans, 9-cis, and 13-cis retinoic acid), etretinate, and others as described in EP-A2-0 379367, U.S. Pat. No. 4,887,805, and U.S. Pat. No. 4,888,342 (the disclosures of which are all incorporated herein by reference), and the dissociating retinoids that are specific for AP-1 antagonism (such as those described by Fanjul, et al. in Nature (1994) 372:104-110). Various synthetic retinoids and compounds having retinoid activity and so expected to be useful in this invention are described in the following U.S. Pat. Nos.: 5,514,825; 5,698,700; 5,696,162; 5,688,957; 5,677,451; 5,677,323; 5,677,320; 5,675,033; 5,675,024; 5,672,710; 5,688,175; 5,663,367; 5,663,357; 5,663,347; 5,648,514; 5,648,503; 5,618,943; 5,618,931; 5,618,836; 5,605,915; 5,602,130; 6,344,561; 6,344,463; 6,342,602; 6,320,047; 6,245,786; 6,235,823; 6,225,494; 6,187,933; 6,177,588; 6,147,224; 6,127,382; 6,124,455; 6,117,987; 6,114,533; 6,093,838; 6,090,826; 6,051,713; 6,048,873; 6,037,488; 5,919,970; 5,917,082; 5,917,048; 5,677,323; 5,677,320. Still other compounds described as having retinoid activity are described in other U.S. Pat. Nos.: 6,344,806; 6,355,446; 6,194,601; 6,083,977; 6,043,279; 6,008,251; 6,004,987; 5,945,561; 5,648,563; 5,648,385; 5,618,839; 5,559,248; 5,616,712; 5,616,597; 5,602,135; 5,599,819; 5,556,996; 5,534,516; 5,516,904; 5,498,755; 5,470,999; 5,468,879; 5,455,265; 5,451,605; 5,343,173; 5,426,118; 5,414,007; 5,407,937; 5,399,586; 5,399,561; 5,391,753; and the like, the disclosures of all of the foregoing and following patents and literature references hereby incorporated herein by reference. Specifically preferred, commercially available retinoids include adapalene (e.g., Differin brand) and tazarotene (e.g., Tazorac brand). The ability of a compound to exhibit retinoid activity can be determined by examining the induction of cytoplasmic retininoic acid binding protein (CRABP) or its DNA (as described in U.S. Pat. Nos. 5,871,909 and 5,654,137, the disclosures of which are incorporated herein by reference).

In addition to the foregoing, derivatives thereof, including esters (e.g., retinyl palmitate), reverse esters, alcohols and alcoholates, acids and acid salts, are suitable, especially those which have a lipophilic character or do not diminish the lipophilicity already present in the molecule.

Of these various retinoids, compounds which have a relatively low molecular weight, and which are more non-polar than polar, are more likely to penetrate the skin, and so are more likely to be useful in practicing the present invention.

Retinoic acid, and likely other retinoids, have been known to enhance penetration of various compounds (such as retinoic acid enhancing the penetration of topical minoxidil for hair growth). Accordingly, the use of retinoic acid (or another penetration-enhancing retinoid) in this invention enhances the penetration of the antifungal, thereby improving the antifungal therapy. The use of exfoliants and hydroxy acids (e.g., alpha-hydroxy acids such as lactic or glycolic) can enhance penetration as well, as well as known penetration enhancers.

Examples of compounds dermatologically acceptable and having antifungal effects, and having or likely to have inhibitory effects on the CYP26-mediated degradation of RA and other retinoids, include azoles, especially triazoles, such as ketoconazole (U.S. Pat. Nos. 4,144,346 and 4,223,036), fluconazole (U.S. Pat. No. 4,404,216), itraconazole (U.S. Pat. No. 4,267,179), terconazole, econazole, miconazole, clotrimazole, voriconazole, sertaconazole, and the like, and compatible mixtures thereof.

It would also be beneficial to use such CYP-26 inhibitors in combination with a reduced amount of retinoid; the CYP-26 inhibitor decreases the metabolic elimination of the retinoid and so less retinoid is needed to achieve the same skin color-affecting result. Still further, analytical methods are available for determining whether a given compound inhibits the degradation of RA by applying the compound and testing for changes in CRABP (cytoplasmic retinoic acid binding protein), which will have increased levels if the levels of RA are also increased by the topical application of the test compound.

There are other benefits to the presence of the retinoid, in addition to affecting the skin color. Retinoids generally activate the EGFR (epidermal growth factor receptor), which causes propagation of keratinocytes at the bottom layer of the epidermis. Increased turnover of the skin causes these new cells to move upwards, towards the skin surface, which causes desquamation of the top layer of skin cells. Thus, as the old skin cells are sloughed off, the newer skin cells migrating to the surface will have a more normal pigmentation. Sloughing off of the skin cells may likely accelerate the elimination of the fungal elements in the stratum corneum. Yet another advantage to the use of a retinoid for treating tinea versicolor is that retinoids tend to reduce the oiliness of the skin.

A non-CYP-26 inhibiting antifungal can be used in combination with the retinoid, so that the combination therapy of retinoid and antifungal is present. In such a situation, the further addition to the combination of antifungal and retinoid can include a CYP-26 inhibitor (antifungal or non-antifungal) to decrease the degradation of the retinoid in the skin. The anti-pigmentation effect is preferred with a CYP-26 inhibitor (antifungal or not) being present. Suitable non-CYP-26 inhibiting antifungal agents include tolnaftate, sulfur-salicylic acid soaps, diazines such as flucytosine, allylamines such as terbinafine (Lamisil) and naftifine (Naftin); benzylamines such as butenafine (Mentax); ciclopirox (ciclopirox olamine, ethanolamine salt of ciclopirox 1:1); thiabenzadole, zinc pyrithione; selenium sulfide; and various essential oils, such as oil of bitter orange (Ramadan,.W., Int. J Dermatol. 1996 June; 35(6):448-9), the essential oil of Eucalyptus pauciflora (Shahi, S. K., et al., Skin Pharmacol. Appl. Skin Physiol., 2000 January-February; 13(1):60-4), ozonized sunflower oil (Menendez, S., et al., Mycoses, 2002 October; 45(8):329-32), oils from Artemisia nelagrica, Caesulia axillaris, Chenopodium ambrosioides, Cymbopogon citratus, and Mentha arvensis (Kishore, N. et al., Mycoses, 1993 May-June; 36(5-6):211-5); and compatible mixtures thereof. Non-antifungal compounds that inhibit CYP-26 include, for example, propranolol (20-100), metaprolol, verapamil, diltiazem, nifedipine, cimetidine, ciprofloxacin, enoxacin, norfloxacin, ofloxacin, pefloxacin, erythromycin, troleandomycin, isoniazid, mexiletine, and dexamethasone.

The present composition is preferably applied topically as a combination of the retinoid and the antifungal, and can be formulated in a shampoo (such as Nizoral brand ketoconazole), cream, lotion, ointment, spray (aqueous or non-aqueous based), or even a powder. Alternatively, the compositions can be used together as separate formulations to tailor the antifungal and/or retinoid to the specific patient, as some patients may be severly infected buy have minimal skin discoloration, or conversely a patient may have a typical infection but significant pigmentation changes due to the infection. Such formulations can have conventional additives, such as preservatives, humectants, odorants (frangrances), colorants, viscosity modifiers, stabilizers, surface active agents, and the like, and mixtures thereof, as conventionally used in such formulations.

For any formulation, each of the components is present in an amount effective to achieve the particular antifungal or skin pigmentation effect. Generally, the antifungal is present in an amount of about 0.2% to 5%, more typically about 0.5% to 3.5%, and most typically about 1-2%. The retinoid is generally present in an amount of about 0.01% to 2%, more typically about 0.02% to 1%. When the retinoid is retinoic acid, it is generally present at 0.025% to 0.1%, and when retinol is the retinoid, it is typically present at 0.1% to 0.25%. A prescription formulation would typically (at present) contain 0.05% retinoic acid and 2% ketoconazole. An exemplary formulation for over-the-counter use would contain 0.25% retinol and 1% ketoconazole. Effective amounts for other retinoids and antifungals are readily determinable by those of ordinary skill in the art. In general, retinol would be used at about two-and-a-half to ten times the concentration of retinoic acid.

The administration of the retinoid and the antifungal can be oral (or systemic) or topical, or a combination. For example, the retinoid can be administered topically while the antifungal is administered orally, or vice versa. Depending on the route of administration, the time of administration may be not the same for both; for example, the retinoid may be administered orally once daily, and the antifungal may be administered topically twice daily.

The foregoing description is meant to be illustrative and not limiting. Various changes, modifications, and additions may become apparent to the skilled artisan upon a perusal of this specification, and such are meant to be within the scope and spirit of the invention as defined by the claims.

What is claimed is:

1. A composition for the treatment of a tinea infection of the skin, consisting essentially of: a combination of an amount of an antifungal agent effective to combat the infection and an amount of a retinoid effective to change the pigmentation of the infected skin, together in a dermatologically acceptable carrier compatible therewith.

2. The composition of claim 1, wherein the retinoid is present in an amount between about 0.01% and about 1%.

3. The composition of claim 1, wherein the antifungal agent is present in an amount between about 0.5% and about 3.5%.

4. The composition of claim 1, wherein the retinoid is selected from the group consisting of retinoic acid, retinol, retinyl esters, and mixtures thereof.

5. The composition of claim 1, formulated as a shampoo or lotion.

6. The composition of claim 1, wherein the antifungal agent is an inhibitor of CYP-26.

7. The composition of claim 6, wherein the antifungal agent is selected from the group consisting of ketoconazole, fluconazole, itraconazole, liarozole, irtemazole, voriconazole, sertaconazole, and combinations thereof.

8. A method for treating a tinea infection of the skin, comprising: topically administering an antifungal compound to the infected area of the skin, and topically administering a retinoid to said infected area of the skin.

9. The method of claim 8, wherein the antifungal and the retinoid are administered concurrently.

10. The method of claim 8, wherein the antifungal compound is an azole.

11. The method of claim 8, wherein the retinoid is selected from the group consisting of retinoic acid, retinol, retinyl esters, and mixtures thereof.

12. The method of claim 8, wherein the antifungal and the retinoid are provided in a single formulation.

13. The method of claim 8, wherein the retinoid is applied as needed to affect the pigmentation of the infected skin and the antifungal is applied on a regular basis.

14. The method of claim 8, wherein the antifungal agent is an inhibitor of CYP-26.

15. The method of claim 14, wherein the antifungal agent is selected from the group consisting of ketoconazole, fluconazole, itraconazole, liarozole, irtemazole, voriconazole, sertaconazole, and combinations thereof.

16. A method for treating tinea versicolor, comprising: administering to a patient in need thereof a combination of a retinoid and an antifungal agent.

17. The method of claim 16, wherein the administration of the retinoid is oral or topical.

18. The method of claim 16, wherein the administration of the antifungal agent is oral or topical.

19. The method of claim 16, wherein the antifungal agent inhibits the breakdown of the retinoid.

20. The method of claim 16, wherein the antifungal agent does not inhibit the breakdown of the retinoid.

21. The method of claim 20, wherein the method further comprises administering a inhibitor compound that inhibits the breakdown of the retinoid.

22. The method of claim 21, wherein the inhibitor compound has antifungal properties.

23. The method of claim 16, wherein the retinoid is retinol or retinoic acid.

24. The method of claim 16, wherein the antifungal agent is selected from the group consisting of ketoconazole, fluconazole, itraconazole, liarozole, irtemazole, voriconazole, sertaconazole, and combinations thereof.
